(19) United States
(12) Patent Application Publication
(10) Pub. No.: US 2016/0206466 A1
(43) Pub. Date: Jul. 21, 2016

(54) UNDERWEAR FOR PELVIC CORRECTION

(71) Applicant: Seung-Ah LEE, Seoul (KR)

(72) Inventor: Seung-Ah LEE, Seoul (KR)

(73) Assignee: DODREAMCREATIVE Co., Ltd., Seoul (KR)

(21) Appl. No.: 14/601,748

(22) Filed: Jan. 21, 2015

(51) Int. Cl. A61F 5/02

(52) U.S. Cl. CPC A61F 5/024; A61F 5/028

(57) ABSTRACT

An underwear for pelvic correction is disclosed, the underwear including: a main body configured to wrap an upper part of a human body; an opening and closing unit including a zipper disposed at a front of the main body; a fastening unit including a plurality of holes disposed at a rear of the main body and a rope and a third fixing unit; a plurality of supports configured to support a spine by being fixed at the main body; and a pelvis supporter disposed at a lower part of the main body and made of an elastic mesh member, and including an opening formed between two of wearing holes for legs of a user to be inserted. According to an exemplary embodiment, the underwear has effects of keeping the balance of the pelvis more efficiently by firmly supporting the core muscles and correcting the pelvis and the spine.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present disclosure relates to underwear for pelvic correction. More specifically, the present disclosure relates to underwear configured to realign a pelvis to a right posture by wearing for a long time.

2. Description of Related Art Including Information Disclosed Under 37 CFR 1.97 and 37 CFR 1.98.

Scoliosis refers to an abnormality where the spine is not in a straight line but in a flexure to a side where one side of the spine is curved convexly and the other side is curved concavely, when viewed from the rear. The abnormal curvature of the spine accompanies a rotation of the spine in the scoliosis, causing an overall distortion of the body including shoulders and pelvis. Recently, as the public concerns for the scoliosis increase, awareness to the scoliosis by the public also increases.

Scoliosis may be divided into three categories based on cause of occurrence: A cause-unknown idiopathic scoliosis; a secondary scoliosis which occurs in connection with other diseases such as spinal tumor, appendicitis, neurofibroma, and polio; and a cause-unknown functional scoliosis which temporarily occurs by poor postures or habits.

In general, the scoliosis may occur in orthograde humans, because the spine is under burden due to the operation of gravity. In addition, people often keep their poor postures for a long time while using PCs or smart phones as these gadgets become popular recently. These poor postures affect their spines and cervical vertebrae to tilt the pelvis thereby causing the scoliosis. Thus, the scoliosis patients tend to increase nowadays.

The pelvis is a skeleton formed of two innominate bones, a sacrum, and a coccyx connecting a spine with two legs. The pelvis is a exceedingly important body organism supporting the weight of upper body and controlling a motion range of a joint. In addition, important joints including hip joints and internal reproductive organs such as viscera, a urinary bladder, and a uterus are located in the pelvis. Thus, the pelvis also functions to protect these organs from external shocks. Therefore, when a structural or functional abnormality occurs in the pelvis, a variety of problems may happen. A wide difference in inclination between left pelvis and right pelvis may cause an imbalance of body shape to generate various pains. The balance between thoracic and lumbar spine may collapse to cause an excessive curvature in the lumbar spine, because heights of left and right pelvises are different. This may lead to diseases such as a scoliosis and a costal exotosis due to a rotary deformation of the centrum. In addition, the tilt of the pelvis may negatively affect femoral region joints and weight dispersion to cause angular deformities such as bowlegs and knock knees. When the angular deformity occurs due to the pelvis abnormality, a degenerative arthritis may develop because the knees are unable to disperse the body weight.

In order to avoid the pelvis tilt, pelvic stable floor muscles should fix the body to keep the balance. The pelvic floor muscle means a line passing through the lower part of the pelvis. However, the pelvic floor muscles are not skeletal muscles to move muscles, but tendons applied to the joints. Thus, people hardly recognize the existence of the pelvic floor muscles in general.

When deformity of the pelvis is prevented, diseases such as a scoliosis can be prevented. Pelvis correction and spine correction are closely related. That is, It is preferable that pelvis correction be performed along with spine correction. The conventional orthosis for scoliosis is formed as a plastic shape. Such orthosis fixes only the spine, but does not support the pelvis. Thus, the pelvic floor muscles may not be correctly positioned and strengthened. When the pelvis tilt continues, the effect of spine correction may be reduced by half. Therefore, an orthosis capable of correcting the pelvis and the spine together at the same time is required.

In addition, the conventional orthosis for scoliosis functions to keep the correct posture of the spine by wear like armor. However, scoliosis patients are reluctant to wear these orthosises, because the plastic shapes of the orthosises restrict wearer's activity. Furthermore, the conventional orthosis may damage the skin due to friction between the skin and a stiff finish thereof, and show outside the wearing, even when worn inside the clothing.

### BRIEF SUMMARY OF THE INVENTION

The technical challenge which the present disclosure intends to achieve is, to provide an underwear for pelvic correction which is wearable and convenient to wear, and keeps the balance of the pelvis more efficiently by firmly supporting the core muscles and correcting the pelvis and the spine.

In order to accomplish the aforementioned purpose, in one general aspect of the present disclosure, there is provided an underwear for pelvic correction, comprising: a main body configured to wrap an upper part of a human body; an opening and closing means including a zipper disposed at a front of the main body; a fastening unit including a plurality of holes disposed at a rear of the main body and a rope to pass through the plurality of holes and a third fixing unit formed at a lower part of the plurality of holes; a plurality of supports configured to support a spine by being fixed at the main body; and a pelvis supporter disposed at a lower part of the main body and made of an elastic mesh member made by covering a synthetic yarn including 30˜40 parts by weight of Spandex® and 60˜70 parts by weight of nylon with a 70 s˜80 s cotton yarn, and including an opening formed between two of wearing holes for legs of a user to be inserted.

In some exemplary embodiments of the present disclosure, the main body may be made of a synthetic fiber formed as a double fabric including 80˜90 parts by weight of cotton and 10˜20 parts by weight of Coolmax®.

In some exemplary embodiments of the present disclosure, the opening and closing means may include inside thereof a plurality of fixing units configured to distribute pressure over the opening and closing means, and the plurality of fixing units may include a plurality of hooks and may apply a tension to a pelvis when locked.

In some exemplary embodiments of the present disclosure, the hole may be formed with a metallic eyelet, and the plurality of holes may be arranged on both sides opposite to each other in two rows by being spaced apart from a center at a rear of the main body, and the third fixing unit may be formed at a lower part of the holes and includes a plurality of hooks.

In some exemplary embodiments of the present disclosure, the main body may be formed as a double fabric, and the plurality of supports may be fixed by being inserted between the double fabric of the main body.

In some exemplary embodiments of the present disclosure, the plurality of supports may be made of a metallic material, and may include a pair of first supports disposed on both sides of the opening and closing means and by being arranged opposite to each other from a center to an end of the opening and closing means, two pairs of second supports arranged on both sides opposite to each other from an upper part to a lower part of the main body by being spaced apart from the first support at a front of the main body, a third support disposed at a center of a rear of the main body, a pair of fourth supports disposed on both sides of the fastening unit and by being arranged opposite to each other, a pair of fifth supports arranged on both sides opposite to each other by being spaced apart from the fourth support at a rear of the main body, and a pair of sixth supports disposed on both end sides of the main body.

According to an exemplary embodiment of the present disclosure, the underwear has effects of keeping a correct posture and correcting the pelvis, by firmly supporting the muscles surrounding the pelvis and the core muscles.

### BRIEF DESCRIPTION OF THE SEVERAL VIEWS OF THE DRAWINGS

FIG. 1 is a view illustrating a front of the underwear for pelvic correction according to an exemplary embodiment of the present disclosure.

FIG. 2 is a view illustrating a front of the underwear for pelvic correction of which opening and closing means is opened according to an exemplary embodiment of the present disclosure.

FIG. 3 is a view illustrating a rear of the underwear for pelvic correction according to an exemplary embodiment of the present disclosure.

FIG. 4 is a magnified view illustrating a fastening unit of the underwear for pelvic correction according to an exemplary embodiment of the present disclosure.

FIG. 5 is a view illustrating a rear of the underwear for pelvic correction according to another exemplary embodiment of the present disclosure.

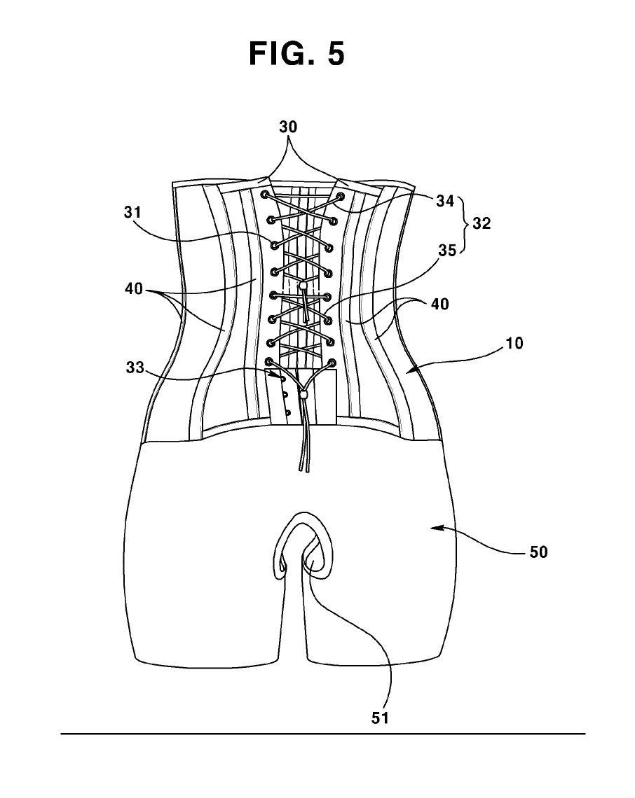

### DETAILED DESCRIPTION OF THE INVENTION

Various exemplary embodiments will be described more fully hereinafter with reference to the accompanying drawings, in which some exemplary embodiments are shown. The present inventive concept may, however, be embodied in many different forms and should not be construed as limited to the exemplary embodiments set forth herein. Rather, the described aspect is intended to embrace all such alterations, modifications, variations, and equivalents that fall within the scope and novel idea of the present disclosure.

The terms including ordinal numbers such as “first” or “second” may be used for description of various elements. However, the elements shall not be limited by such the terms. The terms are used merely to distinguish a particular element from another element.

When a component is mentioned to be “connected” to or “accessing” another component, this may mean that it is directly connected to or accessing the other component, but it is to be understood that another component may exist in-between. On the other hand, when a component is mentioned to be “directly connected” to or “directly accessing” another component, it is to be understood that there are no other components in-between.

The terms mentioned herein are used merely for description of a particular exemplary embodiment. Thus, they are not intended to limit the scope of the present disclosure. The singular expression includes numeral expression thereof unless it has a clearly different meaning in the context. In the present disclosure, the terms such as “include” or “have” are state that there may be in existence of features, numbers, steps, functions, elements, components described herein, or compositions thereof. Therefore, they shall not be understood as to exclude the possibility of existence or addition of one or more other features, numbers, steps, functions, elements, components described herein, or compositions thereof.

Hereinafter, an embodiment of the present invention will be described in detail, with reference to the accompanying drawings.

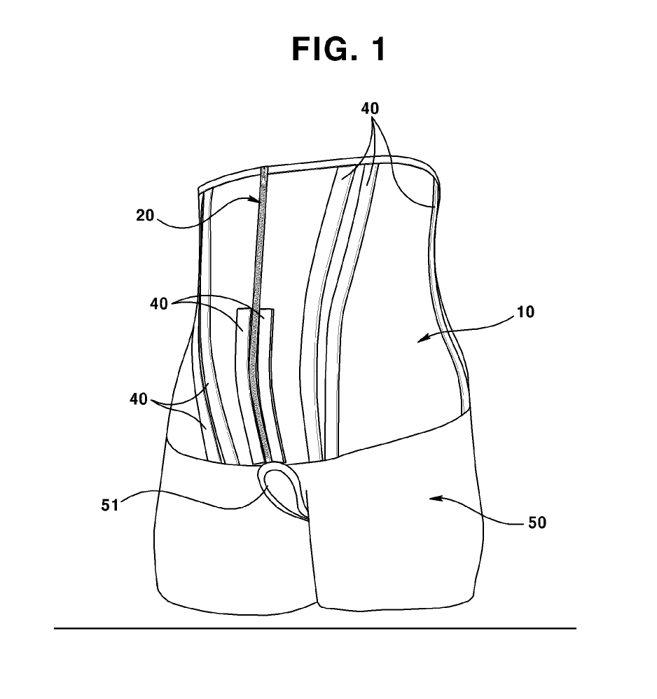

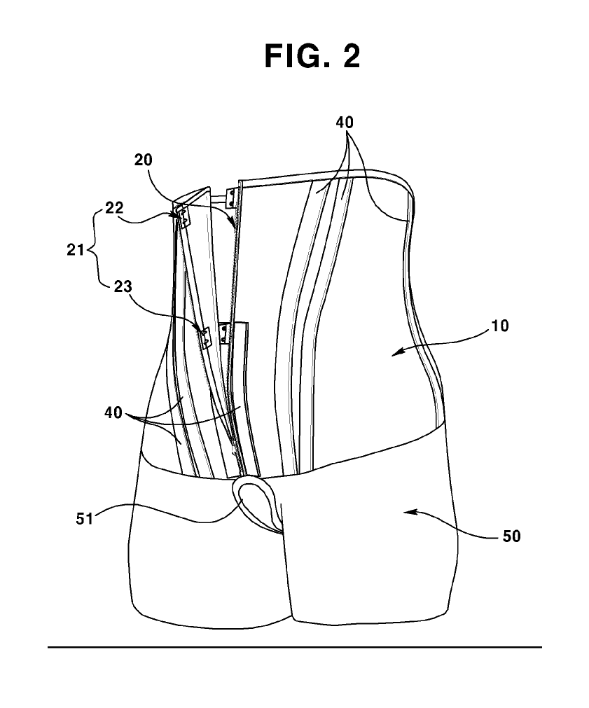

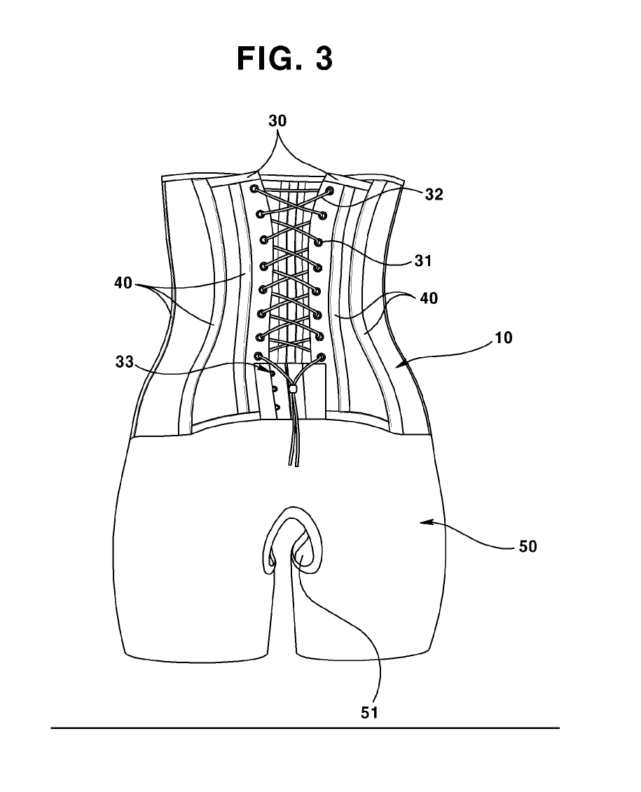

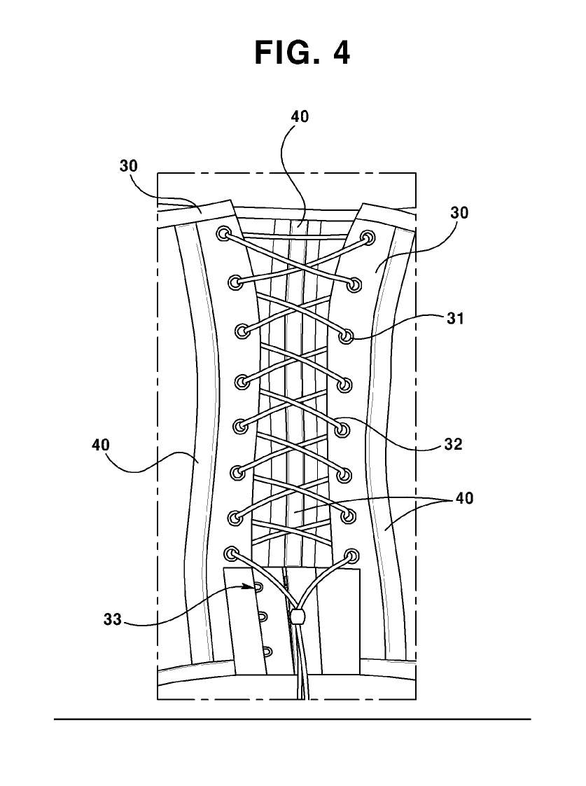

FIG. 1 is a view illustrating a front of the underwear for pelvic correction according to an exemplary embodiment of the present disclosure; FIG. 2 is a view illustrating a front of the underwear for pelvic correction of which opening and closing means is opened according to an exemplary embodiment of the present disclosure; FIG. 3 is a view illustrating a rear of the underwear for pelvic correction according to an exemplary embodiment of the present disclosure; and FIG. 4 is a magnified view illustrating a fastening unit of the underwear for pelvic correction according to an exemplary embodiment of the present disclosure.

Referring to FIGS. 1 and 2, the underwear for pelvic correction according to an exemplary embodiment of the present disclosure may include a main body (10), an opening and closing means (20) disposed at a front of the main body, a fastening unit (30) disposed at a rear of the main body, a plurality of supports (40), and a pelvis supporter disposed at a lower part of the main body.

The main body (10) keeps the upper part of the human body by wrapping and fastening the whole upper and lower abdomen. The main body (10) may be made of a synthetic fiber including 80˜90 parts by weight of cotton and 10˜20 parts by weight of Coolmax®, and may be formed as a double fabric. The coolmax is a functional fabric which is a quick drying fiber developed by DuPont, USA. Such synthetic fiber forming the main body (10) lacks elasticity and is durable so as to firmly support the waist when worn as well as a plastic orthosis. Thus, the main body (10) may provide more comfortable wearing sensation than the conventional plastic orthosis, and at the same time, has an enhanced correction effect compared to the normal corset using Lycra fabric.

The opening and closing means (20) is disposed at a front of the main body, and may be disposed at a center of the front. The opening and closing means (20) may include inside thereof a plurality of fixing units (21). The opening and closing means (20) may be realized as a zipper. The zipper is combined by moving upward from the lower position, so that the underwear according to an exemplary embodiment of the present disclosure may be worn while supporting the abdomen muscles of the user. The fixing unit (21) will be described in detail in the following.

Referring to FIGS. 3 and 4, a fastening unit (30) is formed at a rear of the main body (10). The fastening unit (30) enables the user to adjust the main body (10) to adhere more closely to the body of the user. The fastening unit (30) may include a hole (31), a rope (32), and a third fixing unit (33).

A plurality of the holes (31) may be disposed on both sides of the fastening unit (30) in parallel. The rope (32) fastens the underwear by passing through the plurality of holes (31) to intersect with each other when pulled at the lower end part of the rope (32). The rope (32) may be realized in a line shape to pass through the entire holes (31) by intersecting with each other.

The rope (32) may be formed of a first rope (34) to pass through the upper holes (31) and a second rope (35) to pass through the lower holes (31). It may provide more comfortable wearing sensation to the user by forming the rope (32) of a first rope (34) and a second rope (35), because the tension of the rope (32) affects differently by the status of the abdomen to adjust the pressure on the abdomen.

The third fixing unit disposed at a lower part of the holes (31) may include a plurality of hooks. Thus, when the user wear the underwear using the opening and closing means (20), the underwear for pelvic correction according to an exemplary embodiment of the present disclosure may press and support the user's abdomen more efficiently by fastening the rope (32) of the fastening unit (30). In addition, the fastening unit (30) may distribute the pressure so that different pressures are affecting by positions of the abdomen according to the movement of the user.

In addition, when the third fixing unit (33) is locked, the tension affects the underwear for pelvic correction to wrap the pelvis more firmly. Thus, the spine and the pelvis of the user may be corrected.

Meanwhile, the main body (10) part of the fastening member (30) may be made of an elastic member.

The plurality of supports (40) may be fixed by being inserted between the double fabric of the main body (10). The plurality of supports (40) may support the underwear to adhere closely to the user's body and to keep the correct posture of the spine even when the body is moving. Each of the supports (40) may be inserted by being spaced with a predetermined clearance from each other. The supports (40) may be made of a metallic material, but not limited hereto. Any kind of materials which can apply force and curve by the movement of the body, such as metals, ceramics, plastics, etc., may be included. The supports (40) may be disposed one by one on each of the left and the right side adjacent to the opening and closing means (20) at a front of the main body (10), and may be inserted from a middle part to a lower end of the opening and closing means (20). In addition, pairs of the supports (40) may be symmetrically disposed on the left and the right side herefrom by being spaced with a predetermined clearance. The pairs of the supports (40) may be of a length from an upper end to a lower end of the main body (10), and may be fixed by a sewing line. In addition, among the plurality of the supports (40), one support may be disposed at a center of a rear of the main body (10), each one on both sides of the holes (31) of the fastening unit (30), and each one on both sides of the fastening unit (30). The supports (40) may be inserted between the double fabric of the main body (10) and may be fixed by a sewing line.

The pelvis supporter (50) may be formed at a lower part of the main body (10) for user legs to be inserted. The pelvis supporter (50) may be made of an elastic mesh member that can press the pelvis without constraining the movement of the legs. The pelvis supporter (50) may be made covering a synthetic yarn including 30˜40 parts by weight of Spandex® and 60˜70 parts by weight of nylon with a 70 s˜80 s cotton yarn. Here, the covering refers to a process of weaving a fabric whereby each of a central part yarn called ‘the core’ and an outside part yarn called ‘the cover’ is respectively entered into the needle hole from a different angle, so that the cover made of cotton forms a contact surface to the skin and the core made of synthetic fiber forms a frame. Spandex® is a general term of a synthetic fiber made of an elastic yarn of polyurethane fabric. Thus, the pelvis supporter (50) may provide a good tactile sensation as a cotton material along with elasticity. The pelvis supporter (50) may support both of the spine and pelvis without constraining the activity of the user, because it is elastic.

The pelvis supporter (50) may wrap and support each of the muscles such as pelvic floor muscle, gluteus maximus, femoral muscle, etc., by its grains. Thus, the pelvis supporter (50) may stabilize the pelvis.

An opening (51) may be formed on the pelvis supporter (50). The breathability of the pelvis supporter (50) may be improved by further including the opening (51). In addition, there may be provided an advantage of reducing the constraint of movement along with better sanitation.

Referring to FIG. 2, a plurality of fixing units (21) may be provided inside of the opening and closing means (20). The plurality of fixing units (21) may assist the opening and closing means (20) by distributing the tension of the opening and closing means (20) so that the underwear for pelvic correction may keep applying force to the abdomen in spite of the pressure of the abdomen.

The first and second fixing units (22, 23) may be formed of a plurality of hooks, and may be fixed by combination of the hook parts.

As illustrated in FIGS. 3 and 4, a plurality of supports (40) and a fastening unit are formed at a rear of the main body (10). The user may support the spine more firmly by fastening the rope (32) of the fastening unit (30) after locking the opening and closing means (20), when the user wears the underwear for pelvic correction according to an exemplary embodiment of the present disclosure. In addition, the holes (31) of the fastening unit (30) may be formed of metallic eyelets, which may press and provide stimulation to the erector spinae muscles when the underwear according to an exemplary embodiment of the present disclosure is worn. The erector spinae muscles refer to the muscles disposed at a rear part of the spine. Many of spinal diseases may be prevented by strengthening the erector spinae muscles. One the supports (40) may be formed at a center in the main body (10) inside of the fastening unit (30). This may be consistent with the spine when the user wears the underwear according to an exemplary embodiment of the present disclosure.

Hereinafter, the following exemplary embodiments will provide more specific and detailed description for the effects of the underwear for pelvic correction according to the present disclosure.

### Exemplary Embodiments

### Spine Correction Results of Examinees According to Wearing of the Underwear for Pelvic Correction

Patients with disorders such as scoliosis were instructed to wear the underwear for pelvic correction according to an exemplary embodiment of the present disclosure for a predetermined period of time. Physical changes of the patients, such as shoulder inclination, pelvis inclination, leg curvature (left/right), leg length difference, and mediolateral balance index were measured before and after wearing the underwear for pelvic correction.

Examinee 1

Examinee 1 is a 58-year-old female who had suffered from a carpal tunnel syndrome and a backache. She had a difficulty in lying even when she slept because of backaches such as sciatica. In order to release her symptoms, she was instructed to wear the underwear for pelvic correction according to an exemplary embodiment of the present disclosure, while the correction of muscloskeletal balance was being checked.

Her shoulder inclination, pelvis inclination, leg curvature (left/right), leg length difference, and mediolateral balance index were measured before and after wearing the underwear for pelvic correction.

TABLE 1 shows data where the mediolateral balance index of examinee 1 measured from a front before wearing the underwear, and TABLE 2 shows data where the mediolateral balance index of examinee 1 measured from a front after wearing the underwear for 3 months.

TABLE 3 shows data where the mediolateral balance index of examinee 1 measured from a side before wearing the underwear, and TABLE 4 shows data where the mediolateral balance index of examinee 1 measured from a side after wearing the underwear for 3 months.

TABLE 5 shows data where the mediolateral balance index of examinee 1 measured from a rear before wearing the underwear, and TABLE 6 shows data where the mediolateral balance index of examinee 1 measured from a rear after wearing the underwear for 3 months.

TABLE 1

| | | Inclination | Inclination |
|---|---|---|---|
| Measurement Item | Direction | Angle | Difference |
| shoulder inclination | left up | 0.5 | 2.9 |
| pelvis inclination | right up | 1.5 | 8.8 |
| leg curvature | balanced/o | 1.6/3.7 | −8.9/−20.8 |
| (left/right) | | | |
| leg length difference | right long | | 15.6 |
| mediolateral balance | rightward | left: 47.6% | right: 52.4% |

TABLE 2

| | | Inclination | Inclination |
|---|---|---|---|
| Measurement Item | Direction | Angle | Difference |
| shoulder inclination | right up | 0.0 | 0.2 |
| pelvis inclination | right up | 0.3 | 1.8 |
| leg curvature | balanced/o | 2.7/3.0 | −14.8/−16.4 |
| (left/right) | | | |
| leg length difference | right long | | 7.3 |
| mediolateral balance | rightward | left: 48.4% | right: 51.6% |

TABLE 3

| | | Inclination | Inclination |
|---|---|---|---|
| Measurement Item | Direction | Angle | Difference |
| cervical vertebral | backward | 14.2 | 17.8 |
| inclination | | | |
| upper body | balanced | 22.3 | 107.0 |
| inclination | | | |
| anteroposterior pelvic | forward | 26.6 | 99.7 |
| inclination | | | |

TABLE 4

| | | Inclination | Inclination |
|---|---|---|---|
| Measurement Item | Direction | Angle | Difference |
| cervical vertebral | balanced | 21.4 | 34.3 |
| inclination | | | |
| upper body | balanced | 24.8 | 130.3 |
| inclination | | | |
| anteroposterior pelvic | forward | 15.8 | 53.1 |
| inclination | | | |

TABLE 5

| | | Inclination | Inclination |
|---|---|---|---|
| Measurement Item | Direction | Angle | Difference |
| vertebrae vending | S-character type | 1.3 | 11.9 |
| scapular inclination | right up | 4.4 | 14.7 |
| foot pronation and | pronation/pronation | 3.9/12.3 | −3.0/−8.9 |
| supination | | | |
| sway back | right up | 0.6 | 2.9 |

TABLE 6

| | | Inclination | Inclination |
|---|---|---|---|
| Measurement Item | Direction | Angle | Difference |
| vertebrae vending | S-character type | 1.1 | 7.8 |
| scapular inclination | right up | 1.8 | 5.9 |
| foot pronation and | pronation/pronation | 9.1/13.0 | −6.8/−8.5 |
| supination | | | |
| sway back | left up | 0.3 | 1.5 |

As shown in the above tables, when viewed from the front, the shoulder inclination had changed from 2.9 to 0.2, the pelvis inclination from 8.8 to 1.8, the curvature (left/right) from −8.9/20.8 to −14.8/−16.4, the leg length difference from 15.6 to 7.3, respectively, before and after wearing. That is, from the measured data, the difference of lateral inclination had been reduced, as well as the leg length difference. In addition, the vertebrae inclination of examinee 1 was becoming balanced and the numerical values such as vertebrae vending and inclination were returning to normal, not only as a result of measuring from the front, but also from the side and the rear. That is, it is recognizable that the overall physical measurement index of examinee 1 was becoming balanced, along with improvement of the physical status.

The pelvis inclination status of examinee 1 was remarkably improved from 8.8 to 1.8. That is, examinee 1 was able to keep her body's balance, just wearing the underwear for pelvic correction without any additional treatment.

Examinee 2

Examinee 2 is a female patient who had suffered from a gastrointestinal disorder and a cervical pain. She had a cold abdomen and an upper abdominal obesity. In order to release her symptoms, she was instructed to wear the underwear for pelvic correction according to an exemplary embodiment of the present disclosure, while the correction of muscloskeletal balance was being checked.

Her shoulder inclination, pelvis inclination, leg curvature (left/right), leg length difference, and mediolateral balance index were measured before and after wearing the underwear for pelvic correction.

TABLE 7 shows data where the mediolateral balance index of examinee 2 measured from a front before wearing the underwear, and TABLE 8 shows data where the mediolateral balance index of examinee 2 measured from a front after wearing the underwear for 3 months.

TABLE 9 shows data where the mediolateral balance index of examinee 2 measured from a side before wearing the underwear, and TABLE 10 shows data where the mediolateral balance index of examinee 2 measured from a side after wearing the underwear for 3 months.

TABLE 11 shows data where the mediolateral balance index of examinee 2 measured from a rear before wearing the underwear, and TABLE 12 shows data where the mediolateral balance index of examinee 2 measured from a rear after wearing the underwear for 3 months.

TABLE 7

| | | Inclination | Inclination |
|---|---|---|---|
| Measurement Item | Direction | Angle | Difference |
| shoulder inclination | left up | 1.8 | 8.8 |
| pelvis inclination | right up | 7.0 | 38.1 |
| leg curvature | balanced/O | 3.0/3.6 | −14.9/−17.8 |
| (left/right) | | | |
| leg length difference | left long | | 29.5 |
| mediolateral balance | rightward | left: 47.0% | right: 53.0% |

TABLE 8

| | | Inclination | Inclination |
|---|---|---|---|
| Measurement Item | Direction | Angle | Difference |
| shoulder inclination | right up | 0.9 | 4.0 |
| pelvis inclination | left up | 2.5 | 13.7 |
| leg curvature | O/balanced | 2.7/3.0 | −17.8/−3.0 |
| (left/right) | | | |
| leg length difference | left long | | 6.9 |
| mediolateral balance | leftward | left: 51.7% | right: 48.3% |

TABLE 9

| | | Inclination | Inclination |
|---|---|---|---|
| Measurement Item | Direction | Angle | Difference |
| cervical vertebral | straight neck | 7.5 | 11.9 |
| inclination | | | |
| upper body | balanced | 24.1 | 121.9 |
| inclination | | | |
| anteroposterior pelvic | forward | 13.9 | 41.1 |
| inclination | | | |

TABLE 10

| | | Inclination | Inclination |
|---|---|---|---|
| Measurement Item | Direction | Angle | Difference |
| cervical vertebral | straight neck | 7.8 | 13.4 |
| inclination | | | |
| upper body | balanced | 24.2 | 124.5 |
| inclination | | | |
| anteroposterior pelvic | forward | 7.9 | 23.0 |
| inclination | | | |

TABLE 11

| | | Inclination | Inclination |
|---|---|---|---|
| Measurement Item | Direction | Angle | Difference |
| vertebrae vending | C-character type | 4.8 | 26.8 |
| scapular inclination | right up | 2.8 | 8.8 |
| foot pronation and | pronation/pronation | 3.6/6.8 | −3.0/−5.9 |
| supination | | | |
| sway back | right up | 1.8 | 8.8 |

TABLE 12

| | | Inclination | Inclination |
|---|---|---|---|
| Measurement Item | Direction | Angle | Difference |
| vertebrae vending | C-character type | 3.1 | 18.0 |
| scapular inclination | right up | 2.8 | 9.4 |
| foot pronation and | balanced/pronation | 0.0/6.4 | 0.0/−5.9 |
| supination | | | |
| sway back | left up | 0.4 | 2.0 |

As shown in the above tables, when viewed from the front, the shoulder inclination had changed from 8.8 to 4.0, the pelvis inclination from 38.1 to 13.7, the curvature (left/right) from −14.9/−17.8 to −17.8/−3.0, the leg length difference from 29.5 to 6.9, respectively, before and after wearing. That is, from the measured data, the difference of lateral inclination had been reduced, as well as the leg length difference. In addition, the vertebrae inclination of examinee 2 was becoming balanced and the numerical values such as vertebrae vending and inclination were returning to normal, not only as a result of measuring from the front, but also from the side and the rear. That is, it is recognizable that the overall physical measurement index of examinee 2 was becoming balanced, along with improvement of the physical status.

That is, examinee 2 was also able to keep her body's balance, just wearing the underwear for pelvic correction without any additional treatment.

Examinee 3

Examinee 3 is a female patient who had had a cold abdominal sensation and a gastrointestinal disorder. She suffered from an abdominal obesity and a backache. In order to release her symptoms, she was instructed to wear the underwear for pelvic correction according to an exemplary embodiment of the present disclosure for about 7 months, while the correction of muscloskeletal balance was being checked.

Her shoulder inclination, pelvis inclination, leg curvature (left/right), leg length difference, and mediolateral balance index were measured before and after wearing the underwear for pelvic correction.

TABLE 13 shows data where the mediolateral balance index of examinee 3 measured from a front before wearing the underwear, and TABLE 14 shows data where the mediolateral balance index of examinee 3 measured from a front after wearing the underwear for 3 months.

TABLE 15 shows data where the mediolateral balance index of examinee 3 measured from a side before wearing the underwear, and TABLE 16 shows data where the mediolateral balance index of examinee 3 measured from a side after wearing the underwear for 3 months.

TABLE 17 shows data where the mediolateral balance index of examinee 3 measured from a rear before wearing the underwear, and TABLE 18 shows data where the mediolateral balance index of examinee 3 measured from a rear after wearing the underwear for 3 months.

TABLE 13

| | | Inclination | Inclination |
|---|---|---|---|
| Measurement Item | Direction | Angle | Difference |
| shoulder inclination | left up | 4.3 | 19.1 |
| pelvis inclination | left up | 3.6 | 18.8 |
| leg curvature | balanced/O | 1.1/4.0 | −4.9/−18.0 |
| (left/right) | | | |
| leg length difference | left long | | 11.6 |
| mediolateral balance | rightward | left: 48.8% | right: 51.2% |

TABLE 14

| | | Inclination | Inclination |
|---|---|---|---|
| Measurement Item | Direction | Angle | Difference |
| shoulder inclination | left up | 2.4 | 10.8 |
| pelvis inclination | left up | 0.3 | 1.6 |
| leg curvature | balanced/balanced | 2.2/2.0 | −10.7/−9.6 |
| (left/right) | | | |
| leg length difference | left long | | 2.0 |
| mediolateral balance | leftward | left: 50.8% | Right: 51.2% |

TABLE 15

| | | Inclination | Inclination |
|---|---|---|---|
| Measurement Item | Direction | Angle | Difference |
| cervical vertebral | backward | 3.5 | 3.4 |
| inclination | | | |
| upper body | balanced | 26.5 | 116.1 |
| inclination | | | |
| anteroposterior pelvic | forward | 18.9 | 58.8 |
| inclination | | | |

TABLE 16

| | | Inclination | Inclination |
|---|---|---|---|
| Measurement Item | Direction | Angle | Difference |
| cervical vertebral | backward | 3.3 | 4.2 |
| inclination | | | |
| upper body | balanced | 23.6 | 109.6 |
| inclination | | | |
| anteroposterior pelvic | forward | 7.7 | 22.0 |
| inclination | | | |

TABLE 17

| | | Inclination | Inclination |
|---|---|---|---|
| Measurement Item | Direction | Angle | Difference |
| vertebrae vending | S-character type | 3.5 | 12.2 |
| scapular inclination | left up | 7.0 | 21.3 |
| foot pronation and | balanced/balanced | 2.6/0.1 | 1.6/−0.1 |
| supination | | | |
| sway back | left up | 3.3 | 13.4 |

TABLE 18

| | | Inclination | Inclination |
|---|---|---|---|
| Measurement Item | Direction | Angle | Difference |
| vertebrae vending | S-character type | 3.9 | 19.8 |
| scapular inclination | left up | 2.2 | 7.0 |
| foot pronation and | pronation/balanced | 5.1/0.4 | −3.3/−0.2 |
| supination | | | |
| sway back | left up | 2.5 | 12.6 |

As shown in the above tables, when viewed from the front, the shoulder inclination had changed from 19.1 to 10.8, the pelvis inclination from 18.8 to 1.6, the curvature (left/right) from −4.9/−18.0 to −10.7/−9.6, the leg length difference from 11.6 to 2.0, respectively, before and after wearing. That is, from the measured data, the difference of lateral inclination had been reduced, as well as the leg length difference. In addition, the vertebrae inclination of examinee 2 was becoming balanced and the numerical values such as vertebrae vending and inclination were returning to normal, not only as a result of measuring from the front, but also from the side and the rear. That is, it is recognizable that the overall physical measurement index of examinee 2 was becoming balanced, along with improvement of the physical status.

That is, examinee 3 was also able to keep her body's balance, just wearing the underwear for pelvic correction without any additional treatment.

As described in the above, the underwear for pelvic correction according to an exemplary embodiment of the present disclosure is convenient to wear and provides a good wearing sensation. In addition, the underwear for pelvic correction according to an exemplary embodiment of the present disclosure enables the user to keep a correct posture and balance of the body by firmly supporting the core muscles.

The abovementioned exemplary embodiments are intended to be illustrative, and not to limit the scope of the claims. Many alternatives, modifications, variations, and equivalents will be apparent to those skilled in the art. The features, structures, methods, and other characteristics of the exemplary embodiments described herein may be combined in various ways to obtain additional and/or alternative exemplary embodiments. Therefore, the technical scope of the rights for the present disclosure shall be decided by the claims.

I claim:

1. An underwear for pelvic correction, comprising:
a main body configured to wrap an upper part of a human body;
an opening and closing means including a zipper disposed at a front of the main body;
a fastening unit including a plurality of holes disposed at a rear of the main body and a rope to pass through the plurality of holes and a third fixing unit formed at a lower part of the plurality of holes;
a plurality of supports configured to support a spine by being fixed at the main body; and
a pelvis supporter disposed at a lower part of the main body and made of an elastic mesh member made by covering a synthetic yarn including 30˜40 parts by weight of Spandex® and 60˜70 parts by weight of nylon with a 70 s˜80 s cotton yarn, and including an opening formed between two of wearing holes for legs of a user to be inserted.

2. The underwear for pelvic correction of claim 1, wherein the main body is made of a synthetic fiber formed as a double fabric including 80˜90 parts by weight of cotton and 10˜20 parts by weight of Coolmax®.

3. The underwear for pelvic correction of claim 1, wherein:
the opening and closing means includes inside thereof a plurality of fixing units configured to distribute pressure over the opening and closing means, and
the plurality of fixing units includes a plurality of hooks.

4. The underwear for pelvic correction of claim 1, wherein:
the hole is formed with a metallic eyelet, and the plurality of holes are arranged on both sides opposite to each other in two rows by being spaced apart from a center at a rear of the main body, and
the third fixing unit includes a plurality of hooks and applies a tension to a pelvis when locked.

5. The underwear for pelvic correction of claim 1, wherein:
the main body is formed as a double fabric, and
the plurality of supports are fixed by being inserted between the double fabric of the main body.

6. The underwear for pelvic correction of claim 1, wherein:
the plurality of supports are made of a metallic material, and include:
a pair of first supports disposed on both sides of the opening and closing means and by being arranged opposite to each other from a center to an end of the opening and closing means,
two pairs of second supports arranged on both sides opposite to each other from an upper part to a lower part of the main body by being spaced apart from the first support at a front of the main body,
a third support disposed at a center of a rear of the main body,
a pair of fourth supports disposed on both sides of the fastening unit and by being arranged opposite to each other,
a pair of fifth supports arranged on both sides opposite to each other by being spaced apart from the fourth support at a rear of the main body, and
a pair of sixth supports disposed on both end sides of the main body.
